(19) United States
(12) Patent Application Publication
(10) Pub. No.: US 2003/0009199 A1
(43) Pub. Date: Jan. 9, 2003

(54) Electrical therapy device

(75) Inventors: Heinrich Reinke, Baiersdorf-Hagenau (DE); Dirk Muessig, West Linn, OR (US); Max Schaldach, Erlangen (DE); Max Schaldach JR., Berlin (DE)

Correspondence Address: HAHN LOESER & PARKS, LLP, TWIN OAKS ESTATE, 1225 W. MARKET STREET, AKRON, OH, 44313, US

(73) Assignee: BOTRONIK Mess-und Therapiegeraete GmBH & Co. Ingenieubuero Berlin

(21) Appl. No.: 10/186,313

(22) Filed: Jun. 28, 2002

(30) Foreign Application Priority Data
Jul. 4, 2001 (DE) 101 32 612.2

(51) Int. Cl. A61N 1/365

(52) U.S. Cl. 607/17

(57) ABSTRACT

An electrical therapy device for a heart has a hemodynamic sensor (1), an electrostimulation device (2) and a control unit (30). The hemodynamic sensor (1) is adapted to detect a peripheral blood flow. In this arrangement, the control unit (30) has a tachyarrhythmia evaluation device (3) and a telemetry receiver (6). The control unit (30) controls the electrostimulation device (2) for stimulating and defibrillating the heart in dependence on the peripheral blood flow which is detected by the hemodynamic sensor (1). In addition a tachyarrhythmia and/or the peripheral hemodynamic effects thereof is detected, wherein the peripheral hemodynamic effects of the tachyarrhythmia are determined on the basis of the peripheral blood flow which is detected by a peripheral hemodynamic sensor (1). The results of detecting a tachyarrhythmia and/or the peripheral hemodynamic effects thereof can be used to defibrillate or stimulate the heart by means of the electrostimulation device (2).

### ELECTRICAL THERAPY DEVICE

The present invention relates to an electrical therapy device for a heart, comprising an electrostimulation device which is adapted to stimulate the heart, a hemodynamic sensor which is adapted to detect a peripheral blood flow, and a control unit which is adapted to control the electrostimulation device in dependence on the peripheral blood flow detected by the hemodynamic sensor.

### BACKGROUND OF THE ART

The aim of a therapy device of that kind is to regulate the cardiovascular capacity and to supply the body with an adequate amount of oxygen-enriched blood. The cardiac delivery capacity or cardiac output and the amount of blood which is pumped out of the heart per unit of time represent parameters of fundamental interest in the case of implantable electrical therapy devices such as for example cardiac pacemakers and defibrillators. Those parameters represent good indices in regard to determining an adequate blood supply.

If accurate measurement of cardiac output is possible, an electrical therapy device can determine whether the cardiovascular system adequately supplies the body with blood. If in contrast the body is not adequately supplied with blood the electrical therapy device can influence the output or pump capacity of the heart by regulating for example the heart rate.

In contrast to a merely physiological increased heart rate as for example in the case of a hemodynamically stable tachycardia tachyarrhythmias represent a problem in terms of the oxygen supply to the body by the cardiovascular system as they can have a negative influence on the blood and oxygen supply. A tachyarrhythmia can be expressed for example by a pathologically increased heart rate with unstable hemodynamics or by a hemodynamically unstable tachycardia. For the purposes of detecting tachyarrhythmias signals which are recorded in the heart, such as for example an intracardiac ECG, are generally evaluated by means of suitable detection algorithms and criteria. As tachyarrhythmias can occur both in the atrium and also in the ventricle suitably matured algorithms and criteria are necessary to arrive at differentiated detection.

Suitable therapies for atrial or ventricular tachyarrhythmias, depending on the respective nature of the tachyarrhythmia detected, represent for example antitachycardiac stimulation or possibly defibrillation of the heart, in each case by means of an electrical therapy unit.

U.S. Pat. No. 5,188,106, to Nappholz, discloses a hemodynamic control device and a method of regulating the flow of blood in the cardiovascular system by means of a control system with feedback. A hemodynamic condition of a patient is determined by means of ultrasound and a regulating parameter for modulating the hemodynamic system using electrical therapy is derived therefrom. The method provides for monitoring heart contraction and the blood flow output of the heart in order to control an implantable cardiac therapy device and to maintain the cardiac output.

Detection of the cardiac output of the heart is implemented in that case using Doppler ultrasonic procedures, wherein a suitable ultrasonic transducer is implanted as a hemodynamic sensor in the right heart chamber and directed to the left ventricle. A hemodynamic control parameter is derived on the basis of the relative changes in the cardiac output in relation to time and used for monitoring and controlling the hemodynamic condition of the patient. In that case the blood flow is influenced by electrical stimulation of the heart by means of an electrostimulation device. Accordingly the features of the classifying portion of claim 1 are known from that publication.

The hemodynamic parameters of the vascular system can be determined not only in the proximity of the heart but also at peripheral regions. For that purpose it is proposed that an ultrasonic transducer is implanted at a suitable location in a vein. The flow of blood in an adjacent artery is measured by means of the ultrasonic transducer. Those measured values in respect of the flow of blood are then taken into consideration for example in controlling the heart rate. Detection of the cardiac and the peripheral hemodynamic parameters therefore serves exclusively for rate adaptation in the above-indicated publications.

It has been found however that some patients occasionally suffer from fainting attacks in spite of being provided with known electrical therapy devices.

Accordingly the object of the present invention is to provide an electrical therapy device which is designed to detect and avoid incidents of that kind.

### SUMMARY OF THE INVENTION

That object is attained by an electrical therapy device of the kind set forth in the opening part of this specification, with the features of the characterising portion of attached claim 1.

In accordance with the invention an electrical therapy device for a heart includes a hemodynamic sensor, an electrostimulation device and a control unit. The hemodynamic sensor is adapted to detect a peripheral blood flow. The control unit controls the electrostimulation device in dependence on the peripheral blood flow which is detected by the hemodynamic sensor. The electrostimulation device serves for stimulation of the heart in accordance with the control by the control unit. Also provided is a detection unit which is connected to the peripheral hemodynamic sensor and adapted to detect a peripheral hemodynamic undersupply. In a particularly preferred configuration a tachyarrhythmia and/or the peripheral hemodynamic effects thereof are detected, in which case the peripheral hemodynamic effects of a tachyarrhythmia are determined on the basis of the peripheral blood flow which is detected by the peripheral hemodynamic sensor. The results of detection of a tachyarrhythmia and/or the peripheral hemodynamic effects thereof are possibly used to defibrillate or stimulate the heart by means of the electrostimulation device.

Accordingly the device is for the first time capable of detecting a peripheral hemodynamic undersupply and by way of linkage of the detection unit to the control unit so controlling the therapy device that the therapy device is operated in a manner which is suited to the therapy of a patient if the therapy device for example is in the form of an implant and is implanted. In the case of a therapy device which is designed to combat tachyarrhythmias it is thus possible to detect not only a tachyarrhythmia but in addition also the hemodynamic action thereof on the peripheral hemodynamics.

The advantages achieved with the present invention are based on the new realisation that temporary peripheral tachyarrhythmias, instead of acting on the entire blood circulatory system, can influence only the peripheral hemodynamics. Temporary variations in the hemodynamics, which only give rise to purely peripheral variations, cannot be detected by the usual cardiac sensors. By virtue of a deficient hemodynamic supply to peripheral organs such as for example the brain, in that case acute deficiency symptoms such as for example a faint can occur.

It has been found to be further advantageous that not just the peripheral effects of a tachyarrhythmia but also the peripheral vasomotor influence is also detected by the peripheral hemodynamic sensor.

Therefore the advantages achieved by the invention are in particular that the negative effects of temporary tachyarrhythmias of low degree, such as for example an undersupply of oxygen, on the patient are reduced so that deficiency symptoms in the peripheral organs can be avoided or reduced.

In a preferred configuration of the invention the hemodynamic sensor is so designed that an impedance of the peripheral blood, which is dependent on the peripheral blood flow, is measured to detect the peripheral blood flow.

Impedance measurement in respect of the peripheral blood therefore represents a simple way of determining the peripheral blood flow which is used in turn to determine the negative effects of tachyarrhythmias on the peripheral hemodynamics.

In a further preferred configuration of the invention the hemodynamic sensor has two mutually spaced measuring electrodes which serve to measure the impedance of the peripheral blood.

In a further configuration of the invention the hemodynamic sensor has a telemetry transmitter and the control unit has a telemetry receiver which are suitable for communicating with each other. In that way the data can be transmitted from the hemodynamic sensor wirelessly to the control unit so that there is no need for wired connecting lines between the control unit and the hemodynamic sensor.

Instead of or in addition to the hemodynamic sensor in the form of the impedance sensor, the hemodynamic sensor can also be adapted to detect blood oxygen saturation by measurement of the absorption of light by the blood in the wavelength range of between about 600 and 700 nm.

A blood pressure sensor for arterial blood pressure, for example in the form of a piezoelectric sensor, is a further alternative embodiment of the hemodynamic sensor.

Alternatively the hemodynamic sensor can also be adapted to detect the blood flow speed either by ultrasonic Doppler measurement or by laser Doppler measurement.

In a further alternative configuration the hemodynamic sensor includes a thermistor for detecting the blood flow by measurement of the dissipation of heat, that is to say cooling of the thermistor by the blood flow.

In a further preferred configuration of the invention the hemodynamic sensor is arranged in the region of the carotid sinus. In that way it is possible to detect and avoid in particular a deficient supply to the brain.

### BRIEF DESCRIPTION OF THE DRAWINGS

Fuller understanding of the present invention will be had when reference is made to the accompanying drawings, wherein identical parts are identified by identical part numbers and wherein:

FIG. 1 shows a schematic block circuit diagram of an electrical therapy device,

FIG. 2 shows a diagrammatic view of an embodiment of a hemodynamic sensor from FIG. 1, and

FIG. 3 shows a detailed block circuit diagram of the tachyarrhythmia evaluation device of FIG. 1.

### DETAILED DESCRIPTION OF A PREFERRED EMBODIMENT

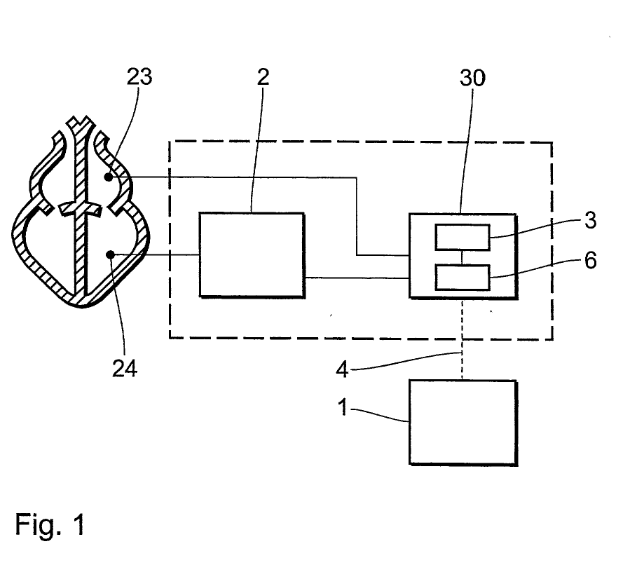

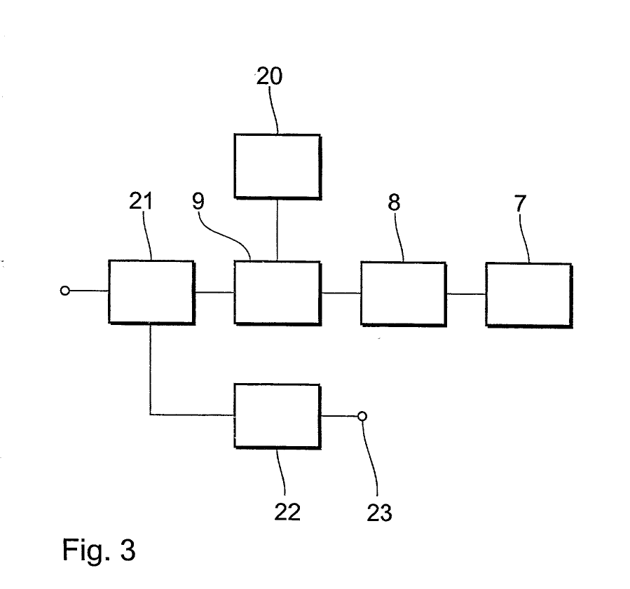

FIG. 1 shows a schematic block circuit diagram of an electrical therapy device. In this case the electrical therapy device comprises a hemodynamic sensor 1, an electrostimulation device 2 and a control unit 30. Both the hemodynamic sensor 1 and also the control unit 30 are adapted to communicate wirelessly with each other, at 4. In this arrangement the control unit 30 has a detection unit 9 (see FIG. 3) which is adapted to analyse a signal originating from the hemodynamic sensor 1, in such a way that an output signal of the detection unit depends on the detection of hemodynamic undersupply as detected by the hemodynamic sensor 1. The therapy device also has a telemetry receiver 6. The electrostimulation device 2 is connected by way of an electrode line to an intracardiac electrode 24 which is used for stimulating and defibrillating the heart. The detection unit 9 is preferably part of an evaluation device 3 which is particularly preferably in the form of a tachyarrhythmia evaluation device 3. The tachyarrhythmia evaluation device 3 is connected to an intracardiac electrode 23 which serves to detect electrical signals of the heart itself, such as for example intracardiac ECG signals. It is basically possible to provide only one electrode which serves both for signal recording and also for delivering stimulation pulses. By way of the electrode 23, the tachyarrhythmia evaluation device is capable of detecting a tachyarrhythmia by assessment of the intracardiac ECG.

By virtue of the detection unit 9, the tachyarrhythmia evaluation device 3 is also capable of linking the information obtained from the intracardiac ECG about the (possible) existence of a tachyarrhythmia with the (possible) existence of a peripheral hemodynamic undersupply. By virtue of suitable AND-linkage of the signals ‘tachyarrhythmia exists’ and ‘peripheral hemodynamic undersupply exists’ the tachyarrhythmia evaluation device 3 can form a control signal for the control unit, which causes the control unit 30 to actuate the electrostimulation device 2 for the output of signals which are suitable for the tachyarrhythmia treatment. Suitable antitachyarrhythmia therapies are basically known. If in the AND-linkage situation (implemented for example by an AND-gate) the signal ‘peripheral hemodynamic undersupply exists’ does not apply the tachyarrhythmia is evaluated as not requiring treatment and a corresponding therapy does not need to be implemented.

Additionally or alternatively the control unit 30 or the tachyarrhythmia evaluation device 3 can also be so designed that a therapy is already triggered off when just a hemodynamic undersupply is detected by the detection unit 9.

While the electrostimulation device 2 and the control unit 30 are implanted in the proximity of the heart the hemodynamic sensor 1 is implanted preferably in the region of the carited sinus. In that case the hemodynamic sensor 1 is implanted within a blood vessel and there detects a peripheral blood flow. Those data are then wirelessly transmitted and received by the control unit 30.

The data received by the control unit 30 are forwarded to the evaluation device 3, the data are evaluated and a check is made on the basis of the electrical heart signals received from the electrode 23 to ascertain whether a tachyarrhythmia situation exists, that is to say a tachyarrhythmia is detected. In addition, on the basis of the received data from the hemodynamic sensor 1, the arrangement detects whether the peripheral effects of the tachyarrhythmia exceed admissible values. As soon as such a tachyarrhythmia is detected, that is to say detection of the tachyarrhythmia and confirmation that admissible values of the peripheral effects thereof are exceeded (more detailed information in this respect is set forth in relation to FIG. 3), the evaluation device 3 initialises the electrostimulation device 2 so that those corresponding countermeasures can be started. Such a countermeasure can represent for example defibrillation or stimulation of the heart by means of the electrode 24. Depending on the respective nature and degree of the tachyarrhythmia detected it can be countered with an adaptedly aggressive therapy.

The values ascertained in respect of the flow of blood can also be additionally used for rate adaptation of the heart, insofar as the electrostimulation device 2 stimulates the heart in dependence on the detected peripheral blood flow.

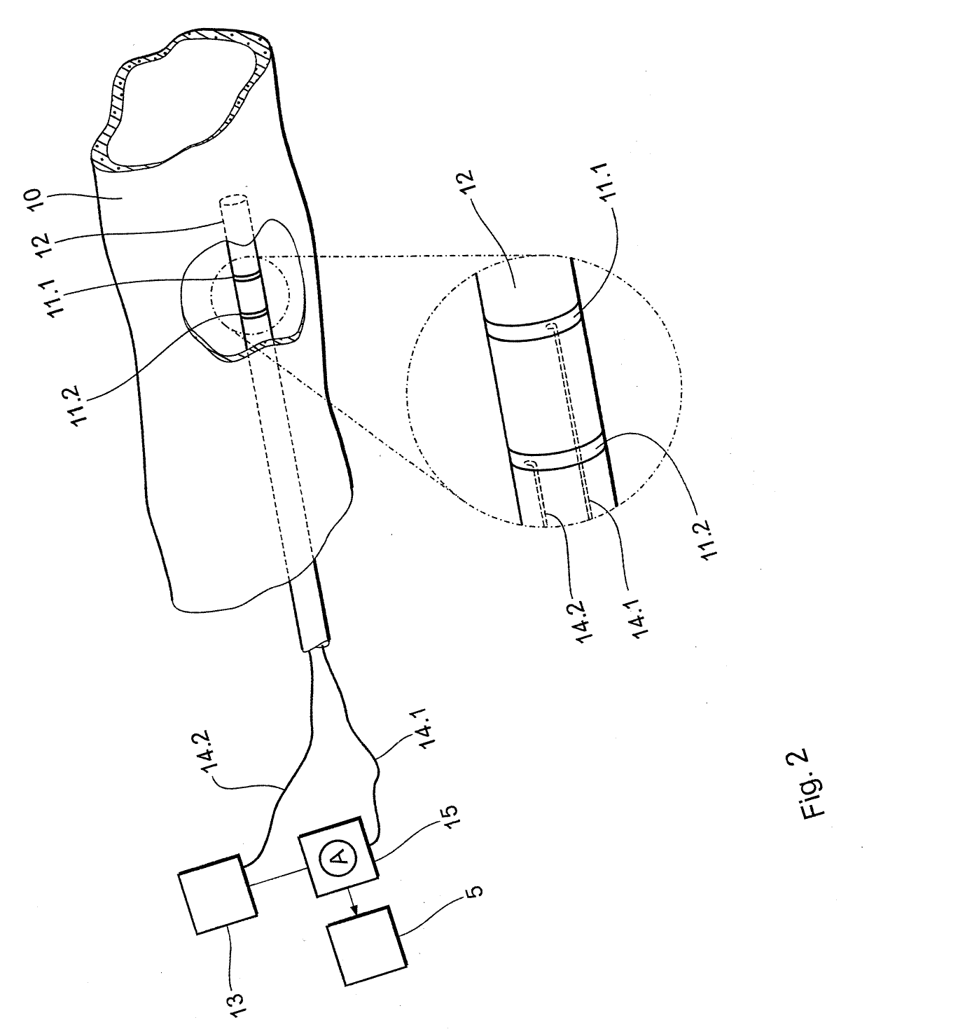

FIG. 2 shows a diagrammatic view of an embodiment of the hemodynamic sensor illustrated in FIG. 1. This hemodynamic sensor is also illustrated in German patent application 196 54 494. The sensor 1 is adapted to measure the impedance of the blood in a blood vessel 10. In that respect, the procedure is based on the surprising realisation that the impedance of the blood depends inter alia on the flow rate of the blood, thereby making it possible to determine the flow of blood in the blood vessel 10. Impedance measurement is effected in this case by means of two measuring electrodes 11.1 and 11.2 which are arranged in the wall of the sensor 12.

For the purposes of measuring impedance, a signal generator 13 which is integrated in the sensor 12 produces an electrical signal which is fed to the two measuring electrodes 11.1 and 11.2 by way of two electrical lines 14.1 and 14.2 which extend in the interior of the sensor 12. A measuring device 15 arranged in the circuit between the signal generator 13 and the two measuring electrodes 11.1 and 11.2 measures the electrical current which flows by way of the two measuring electrodes 11.1 and 11.2. The signal obtained in that way is then fed to a telemetry transmitter 5 where the data are converted in order to be wirelessly transmitted to the control unit 30.

FIG. 3 shows a detailed block circuit diagram of the tachyarrhythmia evaluation device 3. In this case the tachyarrhythmia evaluation device 3 has an impedance calculation means 7, a blood flow calculation means 8, a flow assessment means 9 and a tachyarrhythmia recognition means 21 which are suitably arranged in series with each other. The tachyarrhythmia evaluation device 3 also has a memory 20 which is connected to the flow assessment means 9, and a cardio-detection means 22 connected to the tachyarrhythmia recognition means 21.

The data communicated by the telemetry transmitter 5 are received by the telemetry receiver 6 of the control unit 30. Those data are forwarded to the impedance calculation means 7. There, the impedance of the blood is then calculated from the data in respect of the current flow, by way of the two measuring electrodes 11.1 and 11.2. The blood impedance data are outputted to the blood flow calculation means 8. The blood flow in the blood vessel 10, which reflects the heart time volume, is calculated in dependence on the impedance data. The blood flow data are outputted to the flow assessment means 9. There the blood flow data are compared to blood flow patterns stored in the memory 20. On the basis of the comparison procedure, the arrangement determines whether the blood flow assumes critical values, for the oxygen supply. If that is the case the flow assessment means 9 outputs a corresponding signal to the tachyarrhythmia recognition means 21.

Temporary variations can also be easily detected by continuous detection of the flow of blood in the blood vessel 10. If those variations in the flow of blood deviate considerably from the normal values, that can be caused by virtue of a tachyarrhythmia. Possibly locally limited effects of a tachyarrhythmia in that way can result in an acutely deficient supply of oxygen to peripheral organs such as for example the brain. In extreme cases that deficiency of supply can result in a faint. That is obviated by the electrostimulation device 2 suitably defibrillating or stimulating the heart.

The cardio-detection means 22 detects the electrical signals of the heart itself, such as for example intracardiac ECG signals, by means of the electrode 24, and serves in particular for the detection of tachyarrhythmias. The detected electrical signals of the heart itself are forwarded to the tachyarrhythmia recognition means 21. There any tachyarrhythmias which may occur are recognised and the electrostimulation device 2 is initiated by the tachyarrhythmia recognition means 21 to stimulate or defibrillate the heart in order to reduce the tachyarrhythmia which has occurred.

If the tachyarrhythmia recognition means 21 receives from the cardiodetection means 22 a signal which indicates that there is only a tachyarrhythmia of low level it can happen that the recognition means 21 does not initiate defibrillation or stimulation of the heart. If however in parallel therewith the recognition means 21 receives from the flow assessment means 9 a signal which indicates that the peripheral blood flow is assuming critical values, in terms of oxygen supply, the recognition means 21 will nonetheless initiate defibrillation or stimulation of the heart. In other words, a tachyarrhythmia is detected by the cardio-detection means 22 and the negative effects of the tachyarrhythmia are confirmed by the flow assessment means 9. The initiated defibrillation or stimulation effect by the electrostimulation device 2 is in that case such that it damps the temporary tachyarrhythmia and reduces or eliminates the peripheral effects thereof.

Positioning the hemodynamic sensor 1 in the periphery of a body of a patient, such as for example at the carotid sinus, provides that both the peripheral effects of a tachyarrhythmia and also the peripheral influence of the vasomotor system is detected. In contrast detection of the hemodynamics in the proximity of the heart is not suitable for detecting in particular the peripheral influence of the vasomotor system. In that respect vasomotor influences can interfere with the effects of a tachyarrhythmia and result in compensation for or intensification of the peripheral effects of tachyarrhythmia. Particularly under the influence of alcohol, the vasomotor system can be influenced in such a way that the blood cells are dilated.

In an alternative embodiment it is also possible to determine whether a tachyarrhythmia is present and is to be suitably subjected to therapy, solely on the basis of the blood flow values detected by the hemodynamic sensor, that is to say solely on the basis of the peripheral effects of a possible tachyarrhythmia. In this embodiment the cardio-detection means 22 can be omitted as no electrical signals from the heart are required.

The hemodynamic sensor 1 can also be in the form of a pressure or flow sensor.

In addition hemodynamic sensors 1 can also be arranged at locations other than in the region of the carotid sinus for monitoring the blood flow in the periphery, such as for example the extremities and the head in order to be able to effectively detect and compensate for peripheral tachyarrhythmias.

What is claimed is:

1. An electrical therapy device for a heart, comprising:
a hemodynamic sensor for detecting a peripheral blood flow;
a control unit for emitting a control signal based upon the peripheral blood flow detected by the hemodynamic sensor; and
an electrostimulation device for stimulating the heart based upon the control signal from the control unit;
wherein the control unit comprises a detection unit for a peripheral hemodynamic undersupply, the detection unit being adapted to detect a peripheral hemodynamic undersupply based on the peripheral blood flow detected by the hemodynamic sensor; and
wherein the electrostimulation device is further adapted to deliver signals suitable for defibrillation or stimulation of a heart based upon the peripheral hemodynamic undersupply detected by the detection unit.

2. The electrical therapy device of claim 1, wherein the control unit comprises:
a tachyarrhythmia evaluation device or is connected thereto, such that the tachyarrhythmia evaluation device is connected to the detection unit and is adapted to detect a tachyarrhythmia and/or the peripheral hemodynamic effect thereof, and
wherein the electrostimulation device is further adapted to deliver signals suitable for defibrillation or stimulation of a heart in dependence on the tachyarrhythmia detected by the tachyarrhythmia evaluation device and/or the peripheral hemodynamic effect thereof.

3. The electrical therapy device of claim 2, wherein:
the tachyarrhythmia evaluation device is connected to at least one electrode suitable for recording an intracardiac ECG and is adapted to detect a tachyarrhythmia by evaluating a signal originating from the electrode.

4. The electrical therapy device of claim 3, wherein the tachyarrhythmia evaluation device is adapted to link the electrode signal with the detection unit signal and to form an output signal which is dependent on both input signals.

5. The electrical therapy device of claim 4, wherein:
the hemodynamic sensor is adapted to measure an impedance of the peripheral blood, which is dependent on the peripheral blood flow, in order to detect the peripheral blood flow.

6. The electrical therapy device of claim 1, wherein:
the hemodynamic sensor is adapted to measure an impedance of the peripheral blood, which is dependent on the peripheral blood flow, in order to detect the peripheral blood flow.

7. The electrical therapy device of claim 5, wherein:
the hemodynamic sensor comprises a first and a second impedance measuring electrode, in spaced-apart relationship.

8. The electrical therapy device of claim 6, wherein:
the hemodynamic sensor comprises a first and a second impedance measuring electrode, in spaced-apart relationship.

9. The electrical therapy device of claim 7, wherein the control unit further comprises a telemetry receiver and the hemodynamic sensor further comprises a telemetry transmitter for telemetric communication therebetween, so that peripheral blood flow data detected by the hemodynamic sensor can be wirelessly transmitted to the control unit.

10. The electrical therapy device of claim 8, wherein the control unit further comprises a telemetry receiver and the hemodynamic sensor further comprises a telemetry transmitter for telemetric communication therebetween, so that peripheral blood flow data detected by the hemodynamic sensor can be wirelessly transmitted to the control unit.

11. The electrical therapy device of claim 1, wherein the control unit further comprises a telemetry receiver and the hemodynamic sensor further comprises a telemetry transmitter for telemetric communication therebetween, so that peripheral blood flow data detected by the hemodynamic sensor can be wirelessly transmitted to the control unit.

12. The electrical therapy device of claim 9, wherein:
the hemodynamic sensor is adapted for arrangement in the region of a user's carotid sinus.

13. The electrical therapy device of claim 10, wherein:
the hemodynamic sensor is adapted for arrangement in the region of a user's carotid sinus.

14. The electrical therapy device of claim 3, wherein:
the hemodynamic sensor is adapted to measure an impedance of the peripheral blood, which is dependent on the peripheral blood flow, in order to detect the peripheral blood flow.

15. The electrical therapy device of claim 7, wherein:
the hemodynamic sensor comprises a first and a second impedance measuring electrode, in spaced-apart relationship.

16. The electrical therapy device of claim 15, wherein:
the control unit further comprises a telemetry receiver and the hemodynamic sensor further comprises a telemetry transmitter for telemetric communication therebetween, so that peripheral blood flow data detected by the hemodynamic sensor can be wirelessly transmitted to the control unit.

17. The electrical therapy device of claim 16, wherein:
the hemodynamic sensor is adapted for arrangement in the region of a user's carotid sinus.

18. The electrical therapy device of claim 3, wherein the control unit further comprises a telemetry receiver and the hemodynamic sensor further comprises a telemetry transmitter for telemetric communication therebetween, so that peripheral blood flow data detected by the hemodynamic sensor can be wirelessly transmitted to the control unit.

19. The electrical therapy device of claim 1, wherein the control unit further comprises a telemetry receiver and the hemodynamic sensor further comprises a telemetry transmitter for telemetric communication therebetween, so that peripheral blood flow data detected by the hemodynamic sensor can be wirelessly transmitted to the control unit.
